(19) United States
(12) Patent Application Publication
(10) Pub. No.: US 2014/0155457 A1
(43) Pub. Date: Jun. 5, 2014

(54) Method of Relieving, Improving, Preventing or Treating Xerostomia

(71) Applicant: SUNBIO INC., Gyeonggi-do (KR)

(72) Inventors: KWANG NHO, Gyeonggi-do (KR); Min Jung AHN, Gyeonggi-do (KR); Byung Hee SOHN, Seoul (KR); Chang Min HYUN, Gyeonggi-do (KR); Jung Hun LEE, Gyeonggi-do (KR)

(21) Appl. No.: 14/046,213

(22) Filed: Oct. 4, 2013

(30) Foreign Application Priority Data
Dec. 5, 2012 (KR) 10-2012-0140049
May 15, 2013 (KR) 10-2013-0054877

(51) Int. Cl. A61K 47/48

(52) U.S. Cl. CPC A61K 47/48876; A61K 47/48215
USPC 514/425; 514/546

(57) ABSTRACT

Provided is a method of relieving, improving, preventing or treating xerostomia including applying an oral cavity composition including a polyethylene glycol (PEG) derivative whose reactor is modified to form a covalent bond with an epithelial cell of the oral cavity. An oral moisturizing effect and moisture persistency are increased and the method is effective against xerostomia due to aging or disease.

### CROSS-REFERENCE TO RELATED APPLICATION

This application claims priority to and the benefit of Korean Patent Application No. 10-2012-0140049, filed Dec. 5, 2012 and Korean Patent Application No. 10-2013-0054877, filed May 15, 2013, the disclosure of which is incorporated herein by reference in its entirety.

### BACKGROUND

1. Field of the Invention

The present invention relates to a method of relieving, improving, preventing, or treating xerostomia including applying an oral cavity composition including a polyethylene glycol derivative whose reactor is modified to form a covalent bond with an epithelial cell of an oral mucous membrane (referred to as “oral epithelial cell) of a target.

2. Discussion of Related Art

Xerostomia is a disease of dryness of an oral mucous membrane associated with a change in the composition of saliva or reduced salivary flow due to various reasons. Saliva provides a lubricative function and protects the mucosal surfaces of the oral dryness. Thus, xerostomia can produce serious negative effects on quality of life of patients, affecting dietary habits, speech, taste and increasing susceptibility to dental caries. Depending on the extent of the disease, this typically includes masticatory disturbance dysmasesis, serious bad breath, burning mouth syndrome, and ulceration of the oral mucous membrane.

Causes for temporary or long-term xerostomia include congenital malformation such as salivary gland agenesis, and autoimmune diseases such as the Sjögren's syndrome and a chronic graft-versus-host disease. Also, various diseases such as psychiatric disorders, diabetes, the hepatitis C virus, chronic renal failure, and AIDS (acquired immune deficiency syndrome) can cause xerostomia. In addition, various other medications, radiotherapy complications, stress, anxiety, depression, high temperature fever, diarrhea, blockage of salivary glands by salivary stones or cancer may cause xerostomia as well. In addition, the rate of patients suffering from discomfort associated with low salivary secretion while sleeping due to a significantly decreased amount of saliva secretion, and having increased saliva secretion by various stimuli when they move after waking up due to old age or climacteric syndromes, approaches 23%, and most patients show symptoms of senility (Thie et al., 2002).

Using parasympathomimetic drugs such as cholinergic agents (Pilocarpine; Salagen® MGI Pharmaceuticals, Minneapolis) and Cevimeline (Evoxac®) 3 to 4 times a day after meals (efficacy duration: 2 to 4 hours) which stimulate secretion of saliva were proposed for the treatment of xerostomia. However, such drugs affect other parts of the body, leading to an increase in tears, nasal discharge, or sweat. Parasympathomimetic drugs are prohibited to be used by a patient of asthma, acute iritis, glaucoma, and can induce side effects on patients with a cardiovascular disease, chronic bronchitis, or chronic obstructive pulmonary disease (COPD).

In addition, acupuncture (Blom M and Lundeberg T, 2000), sugar-free gums, candies and tablets containing xylitol, a lactoperoxidase or a glucose oxidase is suggested to stimulate saliva secretion.

With regard to sugar-free gums, candies and tablets, currently Biotene Dry Mouth Gum (Laclede, Rancho Dominguez, Calif.), XyliFresh (Leaf, Espoo, Finland), Salix Lozenges (Scandinavian Naturals, Perkasie, Pa.), etc. are commercially available. Also, research on chewing gums containing saliva stimulants which have had a consistent effect on stimulating long-term saliva secretion (Aagaard et al., 1992) and saliva substitutes which contain a mucosal component and have had great effects on senile xerostomia (Momm and Guttenberger, 2002) have been published.

To date, various methods for treating xerostomia are being researched, but most of them only have an effect of temporarily relieving discomfort and pain caused by xerostomia, so, it will require much more effort to develop a method capable of sufficiently solving the side effects of xerostomia.

In particular, the number of xerostomia patients has increased due to the rise in the population of the elderly. Xerostomia is caused by the reduction of the amount of the secretion of saliva due to excessive intake of medicine and factors related to chronic illness and, as a result, symptoms such as pain and dryness due to bacterial multiplication, bad breath and stomatitis occur. The development of a non-stimulating product that can maintain a sufficient amount of moisture for a long period for the health of the oral cavities of the elderly.

In the present invention, by overcoming the disadvantages of conventional products that temporarily relieve oral dryness, the product of the present invention was developed to have a hypo-allergenic polyethylene glycol (PEG) derivative as main component that does not cause strong stimulation unlike a menthol, an alcohol or the like. Thus, the product is an oral moisturizing and hygienic composition of a granular or tablet type that is easy to carry around and use increasing oral moisture persistency by forming a covalent bond of an oral mucous membrane with the polyethylene glycol derivative.

### SUMMARY OF THE INVENTION

Until now, for the treatment of xerostomia, a cholinergic agent which is a kind of parasympathomimetic drug, acupuncture, saliva stimulants and saliva substitutes have been used. However, these therapeutic agents and adjuvants have a short duration of efficacy for dry mouth relief and affect other parts of the body, resulting in side effects such as an increase in secretion of nasal discharge and sweat, and therefore they have the disadvantage of limitations in use by some patients of asthma or glaucoma.

Therefore, the present invention is directed to providing a method of relieving, improving, preventing or treating xerostomia.

One aspect of the present invention may provide a method of relieving, improving, preventing or treating xerostomia including applying an oral cavity composition having a moisturizing ability and including a polyethylene glycol derivative capable of binding to an oral epithelial cell as an active component in the oral cavity of a target.

Polyethylene glycol is well known to have a moisturizing effect. However, though the polyethylene glycol is simply included in the oral cavity composition, it is temporarily coated on an oral mucous membrane and washed out by water or saliva. For this reason, it does not show an effect of relief towards xerostomia.

To solve this problem, in the present invention, as represented by the following reaction formula, a part of the terminal reactors of the polyethylene glycol is modified to form a covalent bond with the oral epithelial cell, thereby binding a polyethylene glycol derivative to an oral epithelial cell for a long time.

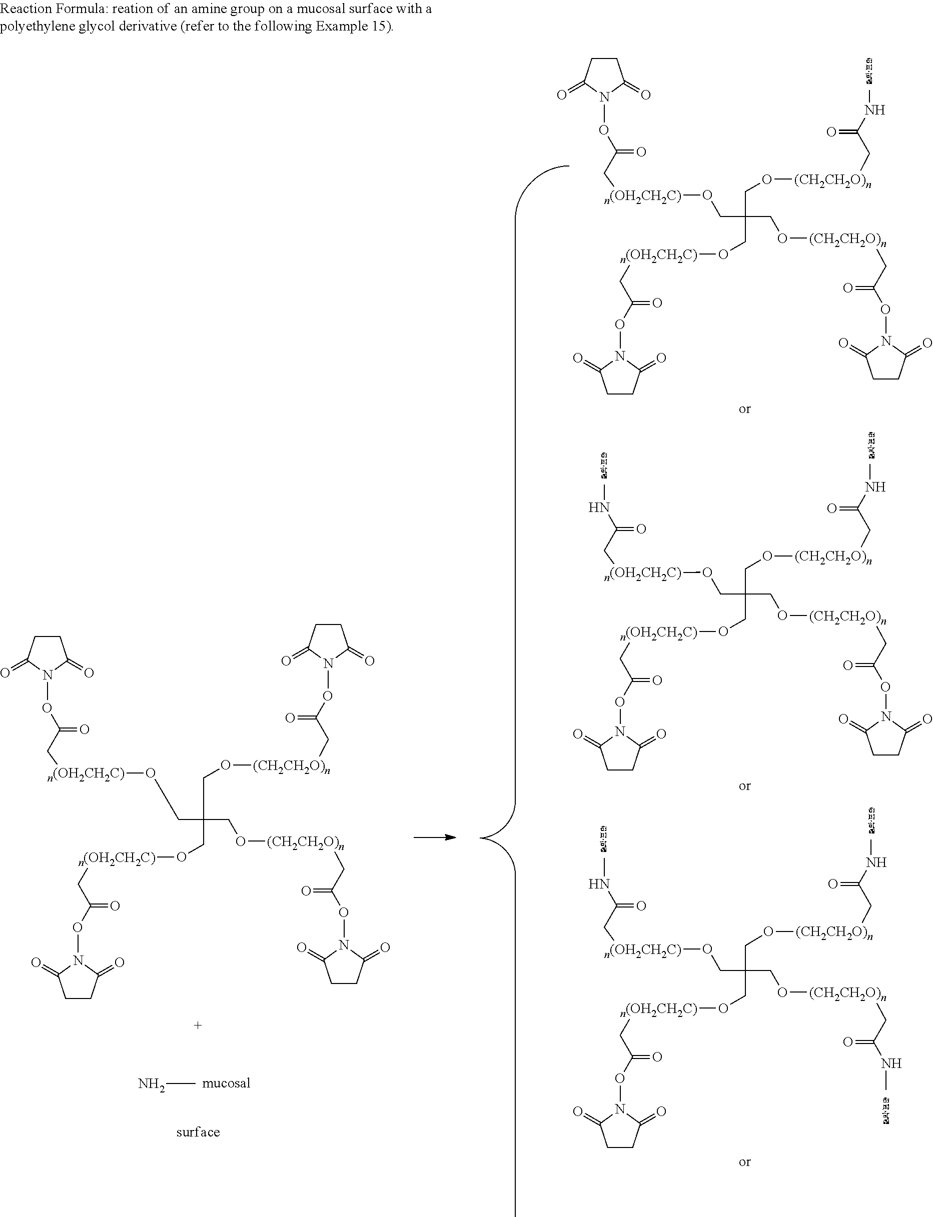

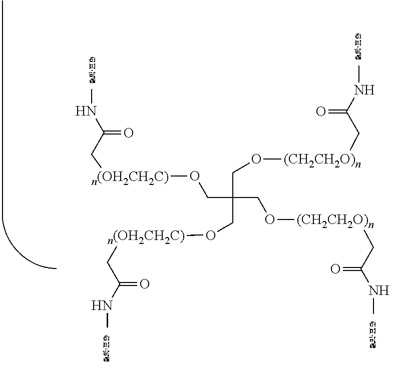

Accordingly, the present invention provides a method of relieving, improving, preventing or treating xerostomia including applying an oral cavity composition having a moisturizing ability and including a polyethylene glycol derivative capable of binding to an oral epithelial cell as an active component in the oral cavity of a target.

In one embodiment of the present invention, the polyethylene glycol derivative whose reactor is modified to form a covalent bond with the oral epithelial cell may form a covalent bond with an amine group present in the oral epithelial cell. Currently, various types of polyethylene glycol derivatives are known in the art and, according to the present invention, when the reactor present at the end of the derivative is modified to a reactor capable of forming a covalent bond with an amine group, the derivative may be used as an active component of the composition.

The reactor may be any one capable of forming a covalent bond with an amine group present in the oral epithelial cell without particular limitation. The reactor may be, but is not limited to, one selected from the group consisting of, for example, halogen, NH₂, SH, CHO,

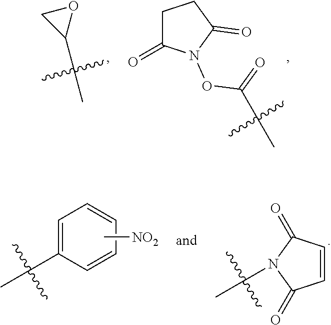

Such a polyethylene glycol derivative may be, but is not limited to, a compound represented by Formula 1 or 2.

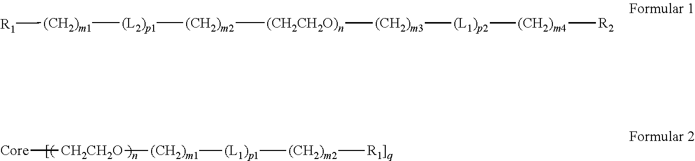

In Formula 1 or 2,

L₁ and L₂ are linkers each independently selected from the group consisting of a carbonyl bond

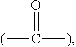

an ester bond

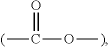

an ester sulfate bond

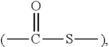

an orthoester bond (orthoester,

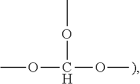

a urethane bond

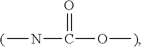

an amide bond

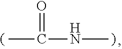

an ester bond (—O—), a carbonate bond

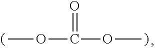

a carbonyl sulfide bond

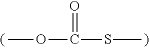

and a secondary amino bond

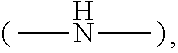

R₁ and R₂ are each independently selected from the group consisting of CH₃O, OH, halogen, NH₂, SH, CHO,

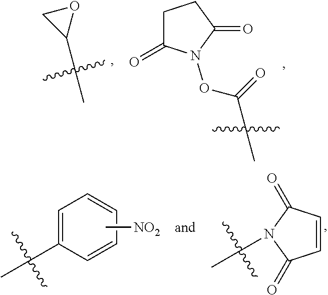

at least one of R₁ and R₂ is not CH₃O or OH,

A core is selected from the group consisting of

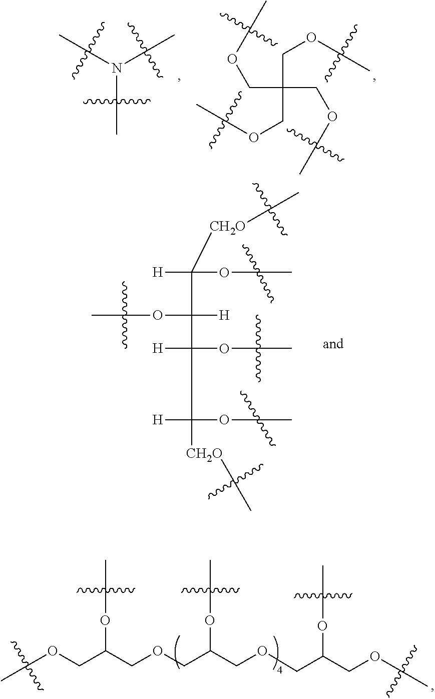

n is an integer from 10 to 2,000,

m₁ to m₄ are each independently an integer from 0 to 3,

p₁ and p₂ are each independently an integer from 0 to 1,

and q is an integer from 3 to 8.

The polyethylene glycol derivative of Formula 1 has a linear structure having one or two reactors modified to react an amine group on the oral epithelial cell with each molecule of the polyethylene glycol, and the polyethylene glycol derivative of Formula 2 is a compound having a cubic structure in which a core is disposed in the center of the molecule, and three, four, six or eight polyethylene glycol derivatives whose reactor is modified to react with an amine group on the oral epithelial cell are bound to the core.

The specific examples of the polyethylene glycol derivative according to the present invention are shown in the following:

### EXAMPLE 1

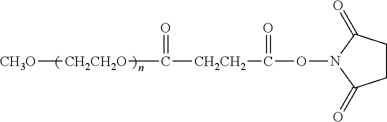

### EXAMPLE 2

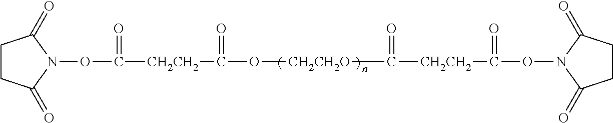

### EXAMPLE 3

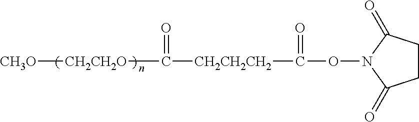

### EXAMPLE 4

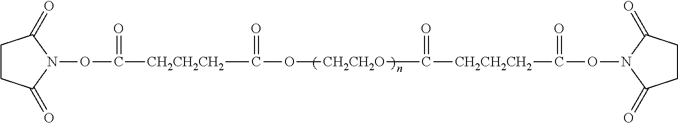

### EXAMPLE 5

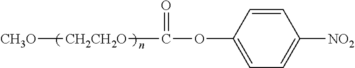

### EXAMPLE 6

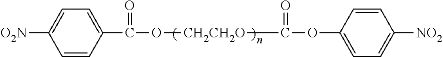

### EXAMPLE 7

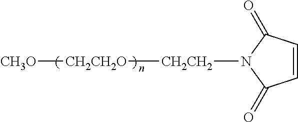

### EXAMPLE 8

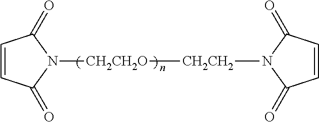

### EXAMPLE 9

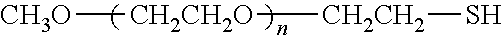

### EXAMPLE 10

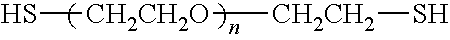

### EXAMPLE 11

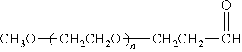

### EXAMPLE 12

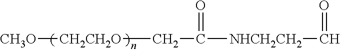

### EXAMPLE 13

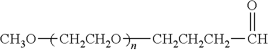

### EXAMPLE 14

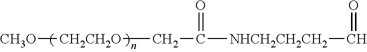

### EXAMPLE 15

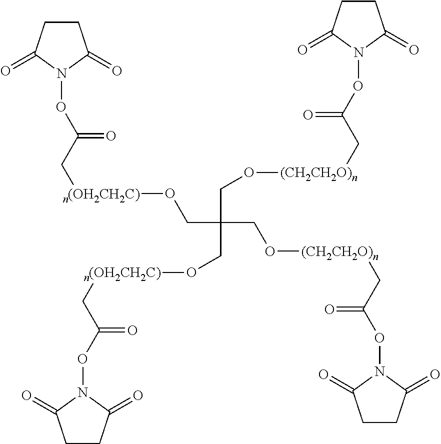

### EXAMPLE 16

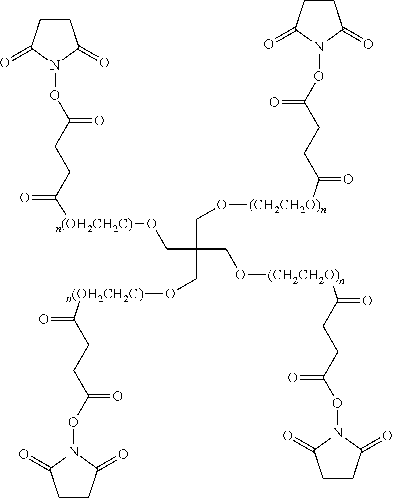

### EXAMPLE 17

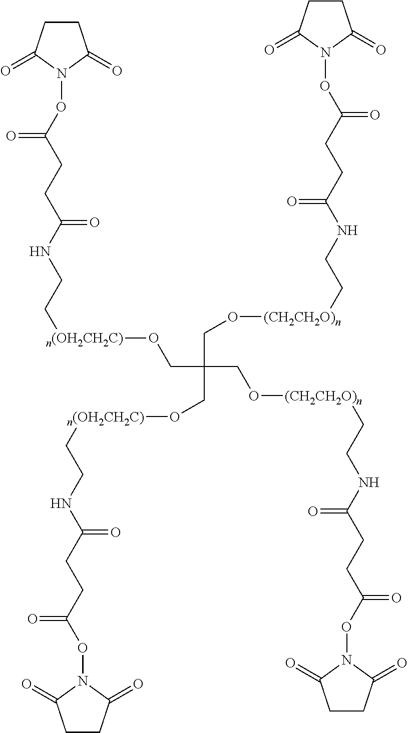

### EXAMPLE 18

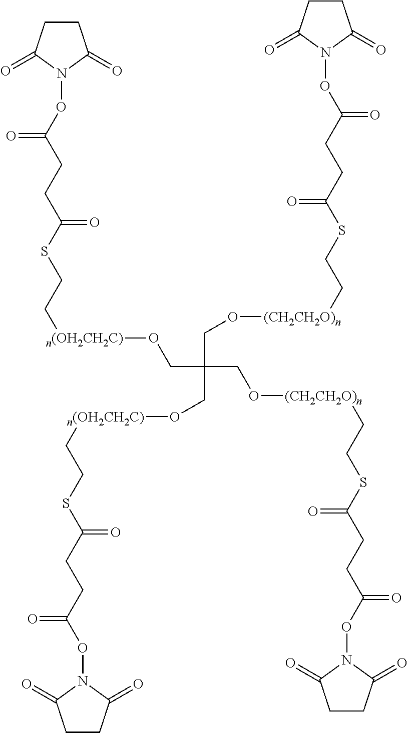

### EXAMPLE 19

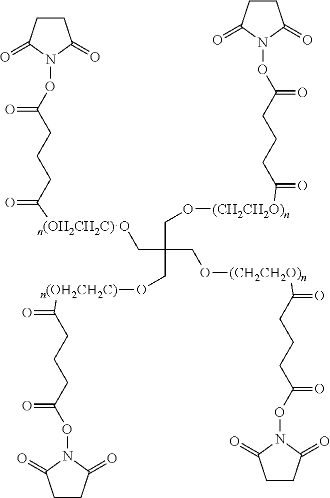

### EXAMPLE 20

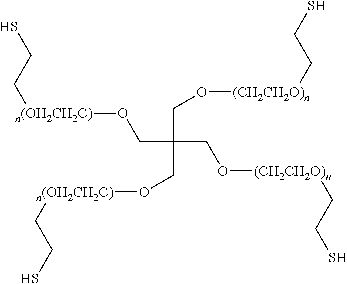

### EXAMPLE 21

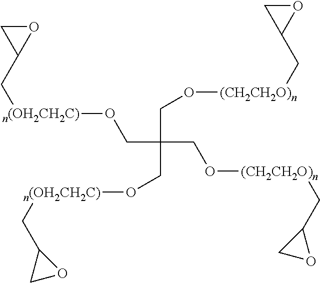

### EXAMPLE 22

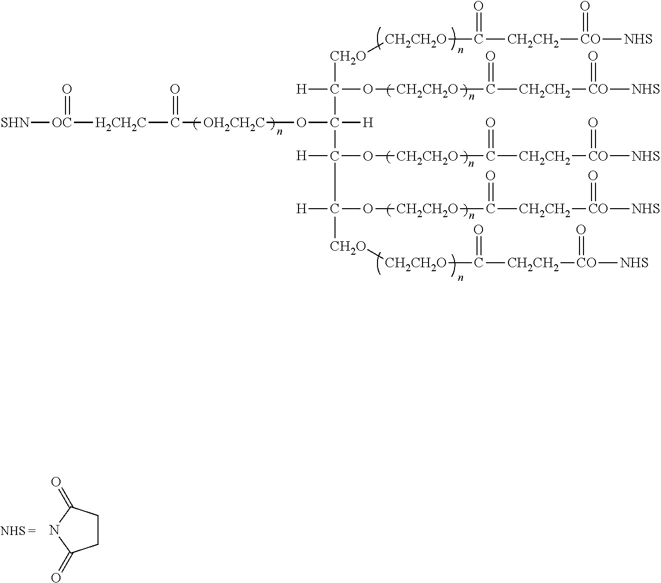

### EXAMPLE 23

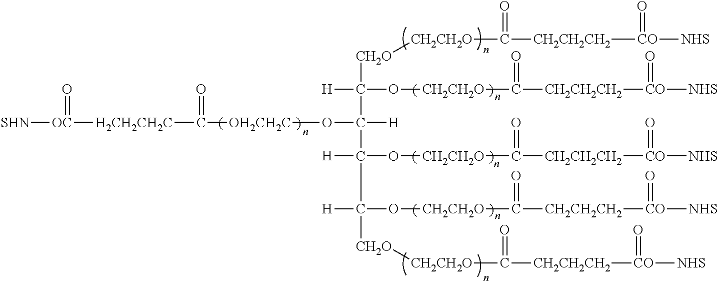

### EXAMPLE 24

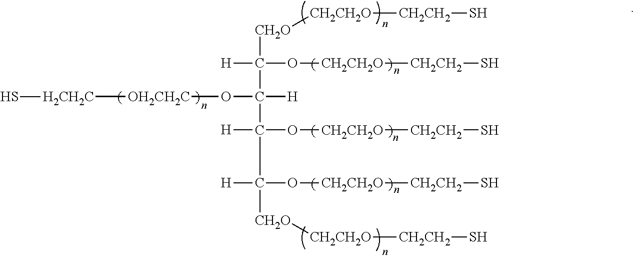

### EXAMPLE 25

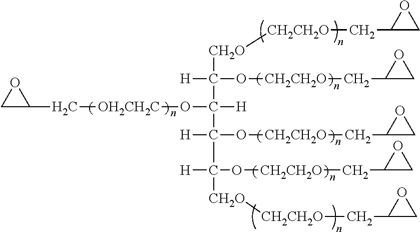

As a method of preparing the polyethylene glycol derivative, Examples 7 and 8 refer to U.S. Pat. No. 6,828,401; Examples 11, 12, 13 and 14 refer to U.S. Pat. Nos. 6,916,962, 6,956,135, 7,041,855, and 7,217,845; and Examples 22, 23, 24 and 25 refer to U.S. Pat. No. 6,858,736. A specific method of preparing a polyethylene glycol derivative according to the present invention will be fully explained in the following Preparation Example.

An oral cavity composition including the polyethylene glycol derivative according to the present invention may be prepared as an oral cavity freshener composition for relieving or improving xerostomia and a pharmaceutical composition for preventing or treating xerostomia.

In addition, the oral cavity freshener composition or medical composition may be formulated as powder or granules.

The oral cavity composition may be blended into a suitable amount with a conventionally used excipient such as a binder, a foaming agent, a sweetening agent, an acidifier, a fragrance, an acidity regulator, etc. according to the purpose of use and the kind of product. Use of the sweetening agent, the acidifier and the fragrance stimulates saliva secretion to increase the duration of moisture in the oral cavity due to binding between a protein contained in saliva or the oral mucosa and the polyethylene glycol derivative and thus increase moisture persistency.

At least one of sorbitol, erythritol, xylitol, lactose, and maltose is added as a sweetening agent to give a sweet taste, and is used in an amount of 20 to 50 parts by weight with respect to a total composition. One or two selected from citric acid, tartaric acid, malic acid, gluconic acid, and succinic acid are mixed as an acidifier for a balanced taste. Orange, lemon, etc. are used as fragrances for food to stimulate a salivary gland and refresh the whole oral cavity to smell good and feel fresh. Sodium hydrogen carbonate is used as an acid regulator to maintain a neutral pH in the oral cavity. In addition, at least one of the pharmaceutically available excipients such as sodium chloride, precipitated calcium carbonate, light anhydrous silicic acid, anhydrous silicic acid, sodium hydrogen carbonate, calcium citrate, DL-alanine, glycine, glucose, D-mannitol, alginic acid, sodium alginate, hydroxypropylmethyl cellulose, methyl cellulose, ethyl cellulose, and sodium carboxymethyl cellulose may be used in addition to the above-described components. The kind and the content of the excipient may be easily determined by one of ordinary skill in the art.

In the present invention, the oral cavity composition may be prepared in a dosage form of powder or granules according to a conventional method.

The powder or granules according to the present invention have no muco adhesive property in a dry state, but as the powders or granules are rapidly gelated by moisture, a mucoadhesive property is recovered. Accordingly, the oral cavity composition of the present invention is a product put in the oral cavity in a suitable amount whenever patients, young or old, feel dryness of the mouth, and is mixed with saliva in the oral cavity or a small amount of water, kept in the oral cavity for approximately 10 to 30 seconds, and spit out. The oral cavity composition moisturizes the oral cavity, prevents tooth cavities, and freshens the oral cavity.

### BRIEF DESCRIPTION OF THE DRAWINGS

The above and other objects, features and advantages of the present invention will become more apparent to those of ordinary skill in the art by describing in detail exemplary the embodiments thereof with reference to the adhered drawings, in which:

FIG. 1 shows images of attachment between a polyethylene glycol derivative of Example 2 and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b);

FIG. 2 shows images of attachment between a polyethylene glycol derivative of Example 4 and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b);

FIG. 3 shows images of attachment between a polyethylene glycol derivative of Example 5 and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b);

FIG. 4 shows images of attachment between a polyethylene glycol derivative of Example 15 and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b);

FIG. 5 shows images of attachment between a polyethylene glycol derivative of Example 19 and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b); and

FIG. 6 shows images of attachment between polyethylene glycol as a control group and oral mucous membrane cells comprising: (a) an image of the fixed Detroit 562 cells obtained by DAPI staining, (b) an image for the polyethylene glycol derivative binding to the outside of the cell membrane obtained by treatment with primary antibody and secondary fluorescent antibody sequentially and (c) a merged image of (a) and (b).

### DETAILED DESCRIPTION OF EXEMPLARY EMBODIMENTS

Hereinafter, exemplary embodiments of the present invention will be described in detail. However, the present invention is not limited to the embodiments disclosed below and can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of the exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to limit the exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, the exemplary embodiments of the present invention will be described in detail below. To aid in understanding the present invention, like numbers refer to like elements throughout the description of the figures, and the description of the same elements will be not reiterated.

Hereinafter, the present invention will be described in detail with reference to the Preparation Examples and the Experimental Examples, but the present invention is not limited thereto.

### 1. Preparation Examples

### (1) Preparation of the Polyethylene Glycol Derivative

### (A) Polyethylene Glycol Derivative of Example 1

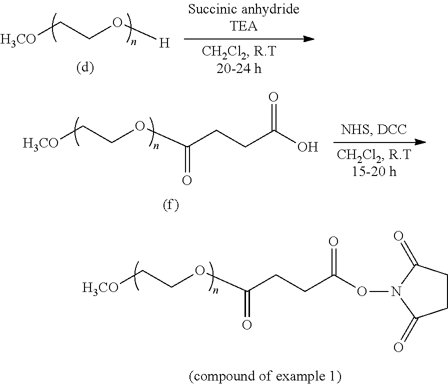

(d) was dissolved in methylene chloride, and triethylamine was added and then stirred at room temperature for 30 minutes. Succinic anhydride was added to the reaction solution and stirred at room temperature for 20 to 24 hours. The reaction solution was washed with distilled water twice. An organic layer solution was dried over magnesium sulfate, concentrated and precipitated with diethylether. A precipitate was filtered, washed with diethylether three times, and dried under vacuum at room temperature for 24 hours, thereby obtaining (f). (f) was dissolved in methylene chloride and N-hydroxysuccinimide (NHS) and dicyclohexyl carbodiimide (DCC) were added in the solution. The reaction was stirred at room temperature for 15 to 20 hours. After the reaction, a byproduct, dicyclohexyl urea (DCU), was filtered using a glass filter, a filtered solution was concentrated, and then the resulting product was precipitated with diethylether. After the precipitate was filtered, it was dissolved in ethyl acetate at 55° C., and recrystallized at 0 to 5° C. for 15 to 17 hours. The recrystallized product was filtered, washed with diethylether three times, and dried under vacuum at room temperature for 24 hours, thereby obtaining a compound of Example 1.

A compound of Example 2 was obtained by using

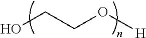

as the raw material (d) in Preparation Example (A).

### (B) Polyethylene Glycol Derivative of Example 3

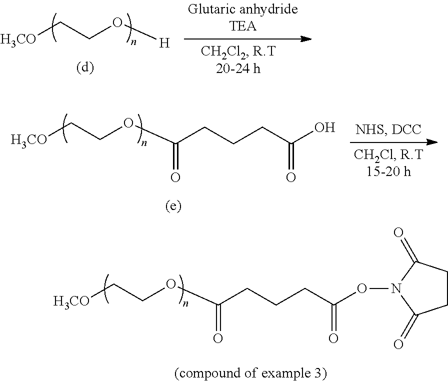

(d) was dissolved in methylene chloride, and triethylamine was added and then stirred at room temperature for 30 minutes. Glutaric anhydride was added to the reaction solution and stirred at room temperature for 20 to 24 hours. The reaction solution was washed with distilled water twice. An organic layer solution was dried over magnesium sulfate, concentrated and precipitated with diethylether. A precipitate was filtered, washed with diethylether three times, and dried under vacuum at room temperature for 24 hours, thereby obtaining (e). (e) was dissolved in methylene chloride and N-hydroxysuccinimide (NHS) and dicyclohexyl carbodiimide (DCC) were added in the solution. The reaction is stirred at room temperature for 15 to 20 hours. After the reaction, dicyclohexyl urea (DCU) which is a byproduct, was filtered using a glass filter, a filtered solution was concentrated, and then the resulting product was precipitated with diethylether. After the precipitate was filtered, it was dissolved in ethyl acetate at 55° C., and recrystallized at 0 to 5° C. for 15 to 17 hours. The recrystallized product was filtered, washed with diethylether three times, and dried under vacuum at room temperature for 24 hours, thereby obtaining a compound of Example 3.

A compound of Example 4 was obtained by using

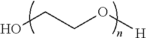

as the raw material (d) in Preparation Example (B).

In addition, the raw material (d) in Preparation Example (B) was used as (a) of Preparation Example (D), thereby obtaining a compound of Example 19.

### (C) Polyethylene Glycol Derivative of Example 5

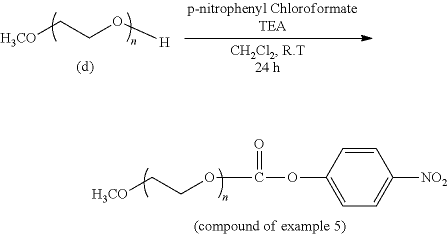

(d) was dissolved in methylene chloride, and then triethylamine was added. Then, p-nitrophenyl chloroformate was added at 0 to 5° C. and stirred at room temperature for 24 hours. The reaction solution was washed with distilled water twice and an organic layer solution was dried over magnesium sulfate, concentrated and precipitated with diethylether. A precipitate was filtered, dissolved in ethyl acetate at 55° C., and recrystallized at 0 to 5° C. for 15 to 17 hours. The recrystallized product was filtered, washed with diethylether three times, and dried under vacuum at room temperature for 24 hours, thereby obtaining a compound of Example 5.

### (D) Polyethylene Glycol Derivative of Example 15

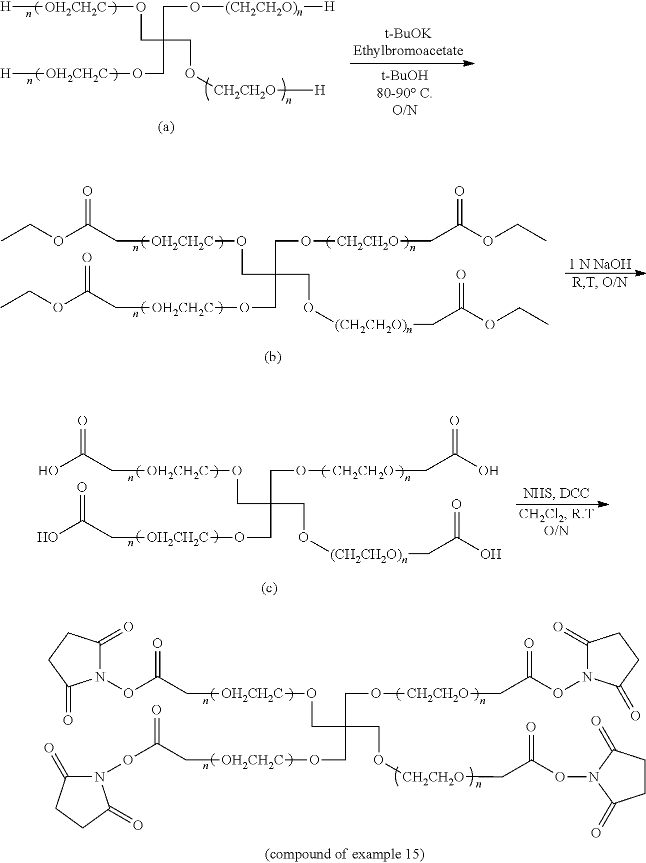

(a) and potassium t-butoxide were dissolved in t-butanol and stirred at 60 to 70° C. for 3 hours. Ethyl bromoacetate was added dropwise and the reaction solution stirred at 80 to 90° C. for 16 to 18 hours. After the reaction solution was concentrated, it was dissolved in methylene chloride. The solution was washed with distilled water, and extracted with methylene chloride. The organic layer solution was dried over magnesium sulfate, filtered, concentrated, and precipitated with diethylether. The precipitate was filtered and dried under vacuum at room temperature for 24 hours, thereby obtaining (b). (b) was dissolved in 1N sodium hydroxide (NaOH) and stirred at room temperature for 16 to 18 hours. The solution was acidified to pH2 with 6N hydrochloric acid (HCl) and extracted with methylene chloride twice. The extracted solution was dried over magnesium sulfate, concentrated, and precipitated with diethylether. The precipitate was filtered and dried under vacuum at room temperature for 24 hours, thereby obtaining (c). (c) was dissolved in methylene chloride, and then N-hydroxysuccinimide (NHS) and dicyclohexyl carbodiimide (DCC) were added. The reaction solution was stirred at room temperature for 15 to 20 hours. After the reaction, dicyclohexyl urea (DCU) which is a byproduct was filtered using a glass filter, a filtered solvent was concentrated, and then the resulting product was precipitated with diethylether. After the precipitate was filtered, dissolved in ethyl acetate at 55° C. and recrystallized at 0 to 5° C. for 15 to 17 hours. The recrystallized product was filtered, washed with diethylether three times, and dried under vacuum at room temperature for 24 hours, thereby obtaining a compound of Example 15.

### (2) Granulation

A powdery polyethylene glycol derivative was granulized to easily use and package. The granulation was performed using a granule former generally used in the pharmaceutical industry.

### (3) Preparation of the Final Dosage Form

A granulized polyethylene glycol derivative (48 parts by weight), erythritol (25 parts by weight), xylitol (16 parts by weight), malic acid (3 parts by weight), lemon flavoring (3 parts by weight), and sodium hydrogen carbonate (5 parts by weight) were well mixed, thereby preparing a final dosage form.

### 2. Experimental Examples

### Confirmation Test for an Attachment of a Polyethylene Glycol Derivative to an Oral Mucous Membrane Cell

Cell lines derived from human pharynx cancer cells, Detroit 562 (ATCC No. CCL138™) cells, were selected to be tested. The cell line was maintained by a monolayer culture in a Dulbecco's modified Eagle's medium (DMEM/F12, Gibco, Calif., USA) including antibiotics (Gibco, Calif., USA) and supplemented by 10% fetal bovine serum (FBS, Gibco, Calif., USA). The cells were cultivated and maintained at 37° C. in 5% CO₂. Cultured cell-lines were maintained for 12 weeks. When the cells were concentrated in a petri dish, the medium was removed, and the remaining cells were washed with phosphate buffered saline (PBS) once, treated with 0.05% trypsin-EDTA (Gibco, Calif., USA), and mixed to be completely separated from the petri dish, and then a DMEM/F12 containing 10% FBS was added to stop the activity of trypsin. Centrifugation was performed at 1000 rpm for 10 minutes, and a supernatant was removed to a cell pellet. The cell pellet was washed with PBS and centrifuged again under the same conditions. A trypsin-EDTA was removed to a cell pellet, a DMEM/F12 containing 10% FBS was mixed, and the mixture was put in a petri dish. The subculture was repeated by the above-described method, and quaternary subcultured cells were used in every experiment. After the drug composition based on the present invention was included in a culture solution to treat the cell line stabilized in the above-described operation, attachment of the dosage form on a surface of the cell was confirmed.

A solution including a polyethylene glycol derivative (the compounds of Examples 2, 4, 5, 15 and 19) and a polyethylene glycol solution used as a control, not a derivative, were each dropped on the Detroit 562 cells stabilized in the above operation by 10% w/v and reacted for 5 minutes. After the reaction solution was washed with PBS, the cultured cells were fixed with 4% para-formaldehyde, and washed with PBS and 0.5% Triton X-100. After washing, the cells were blocked with a solution including 3% bovine serum albumin (BSA) and washed again with PBS several times. After washing, a primary antibody (anti-PEG antibody, rabbit monoclonal antibody, Epitomics, Inc., USA) was added and cultured overnight at 4° C. After the treatment with the primary antibody, the cells were washed with PBS several times and treated with a secondary antibody (fluorescein anti-rabbit IgG, Vector, CA, USA) along with 3% BSA for 4 hours. After the treatment with the secondary fluorescent antibody, the cells were treated with PBS several times and mounted with a mounting medium including DAPI (VECTASHIELD mounting medium with DAPI, Vector, CA USA). After the mounting, using an image restoration phase contrast microscope (Olympus IX70 Inverted Microscope, CA USA), it was confirmed that a polyethylene glycol derivative (the compounds of Examples 2, 4, 5, 15 and 19) was attached to the outside of a cell membrane.

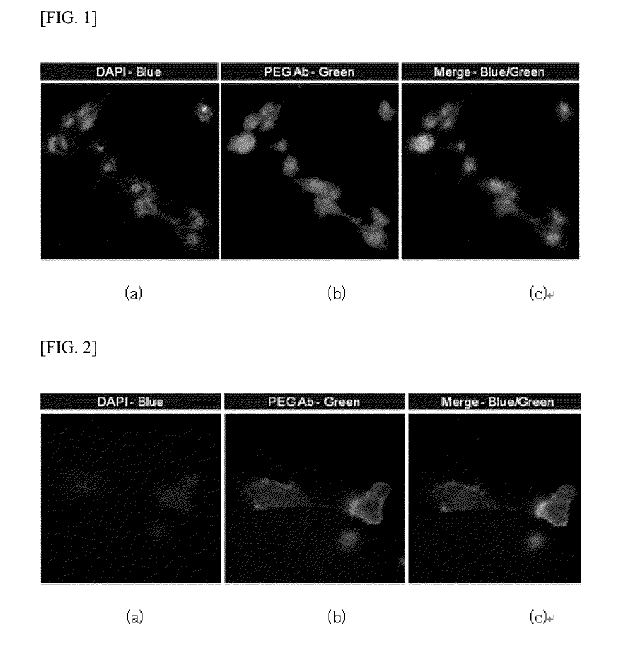

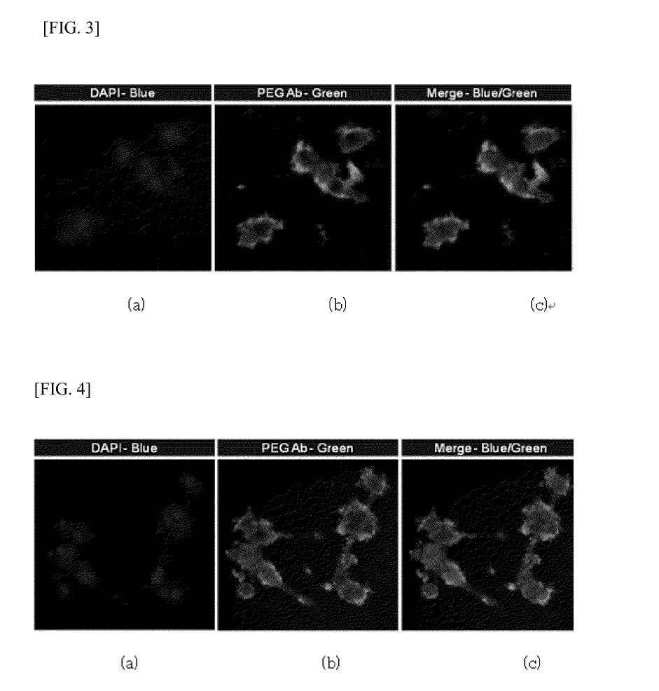

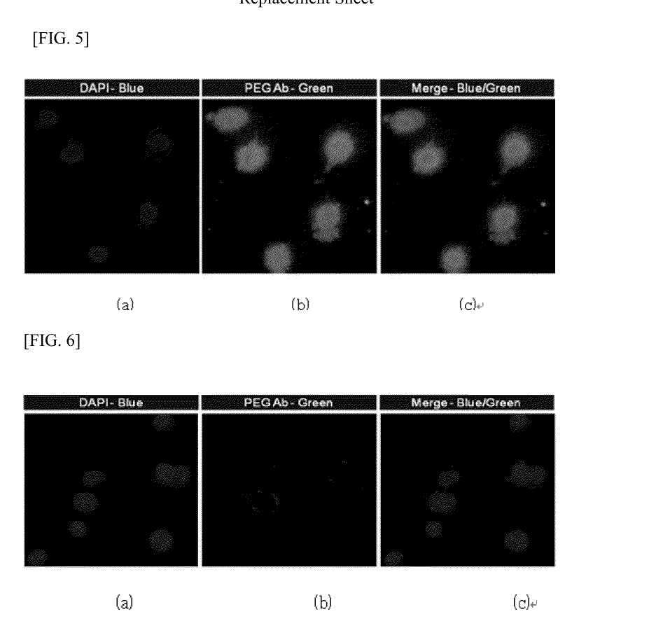

FIGS. 1 to 5 show images of the attachment of polyethylene glycol derivatives of Examples 2, 4, 5, 15 and 19 to oral mucous membrane cells, and FIG. 6 shows images of the attachment of polyethylene glycol as a control, to oral mucous membrane cells.

The presence of the fixed Detroit 562 cells was confirmed using DAPI (4′,6-diamidino-2-phenylindole) staining for dyeing DNA in a cell nucleus (refer to FIGS. 1-(a), 2-(a), 3-(a), 4-(a), 5-(a) and 6-(a)).

The presence of a polyethylene glycol derivative binding to the outside of the cell membrane was confirmed by treatment with the primary antibody recognizing a molecule of the polyethylene glycol derivative and treatment with the secondary fluorescent antibody (refer to FIGS. 1-(b), 2-(b), 3-(b), 4-(b) and 5-(b)).

The presence of the Detroit 562 cells and the polyethylene glycol derivative binding to the outside of the cell membrane thereof were simultaneously observed by projecting overlapping blue and green images using an image restoration phase contrast microscope (refer to FIGS. 1-(c), 2-(c), 3-(c), 4-(c) and 5-(c)). However, it can be confirmed that the polyethylene glycol as a control did not bind to the outside of the cell membrane (refer to FIGS. 6-(a), (b), and (c)).

In a method of relieving, improving, preventing xerostomia according to the present invention, a polyethylene glycol derivative whose reactor is modified to form a covalent bond with an oral epithelial cell is used, resulting in an increase in an oral moisturizing effect and moisture persistency, which is effective against xerostomia due to aging or disease.

While the invention has been shown and described with reference to certain exemplary embodiments thereof, it will be understood by those skilled in the art that various changes in form and details may be made therein without departing from the scope of the invention as defined by the appended claims.

What is claimed is:

1. A method of relieving, improving, preventing or treating xerostomia, comprising:
applying an oral cavity composition including a polyethylene glycol derivative whose reactor is modified to form a covalent bond with a polyethylene glycol derivative as an active component in an oral cavity.

2. The method according to claim 1, wherein the polyethylene glycol derivative has a reactor capable of forming a covalent bond with an amine group on an oral epithelial cell.

3. The method according to claim 2, wherein the polyethylene glycol derivative has a reactor selected from the group consisting of halogen, NH₂, SH, CHO,

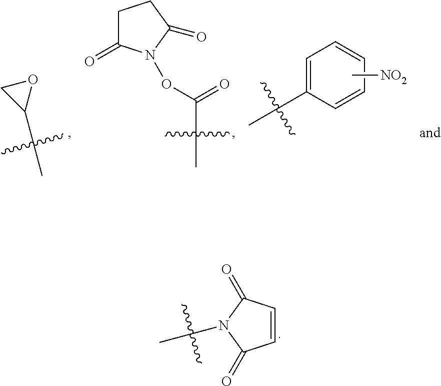

4. The method according to claim 3, wherein the polyethylene glycol derivative is a compound represented by Formula 1 or 2: 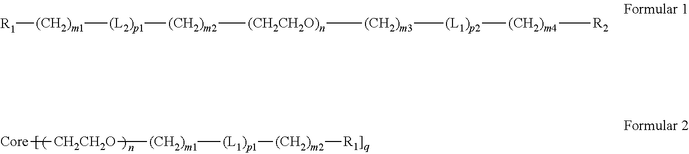
where L₁ and L₂ are linkers which are each independently selected from the group consisting of a carbonyl bond 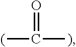
an ester bond 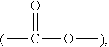
an ester sulfate bond 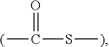
an orthoester bond (orthoester, 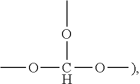
a urethane bond 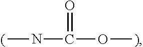
an amide bond 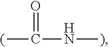
an ester bond (—O—), a carbonate bond 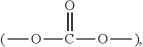
a carbonyl sulfide bond 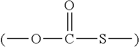
and a secondary amino bond 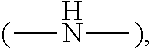
R₁ and R₂ are each independently selected from the group consisting of CH₃O, OH, halogen, NH₂, SH, CHO, 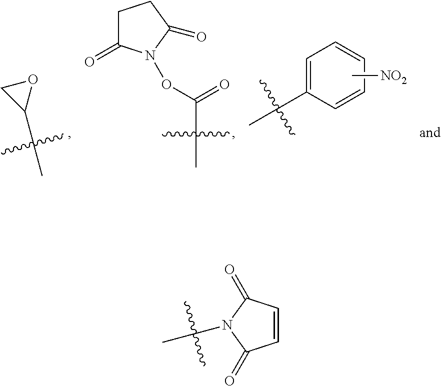
at least one of R₁ and R₂ is not CH₃O or OH, a core is selected from the group consisting of 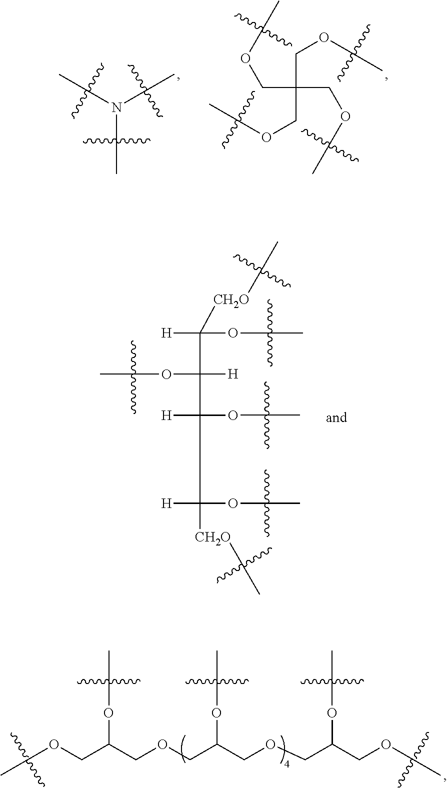
n is an integer from 10 to 2,000, m₁ to m₄ are each independently an integer from 0 to 3, p₁ and p₂ are each independently an integer from 0 to 1, and q is an integer from 3 to 8.

5. The method according to claim 4, wherein the polyethylene glycol derivative is a compound represented by Formula 1 or 2, which is selected from:

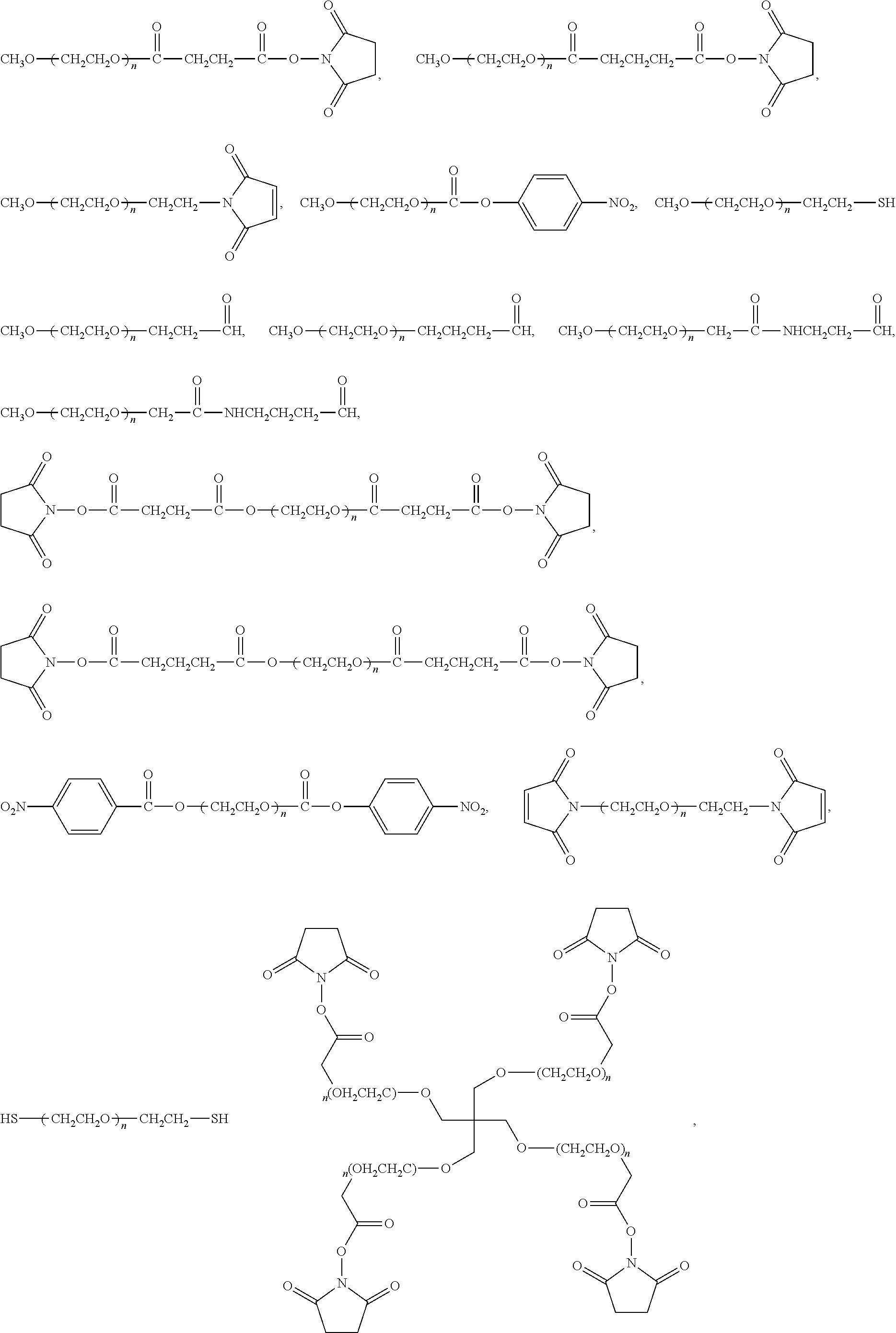

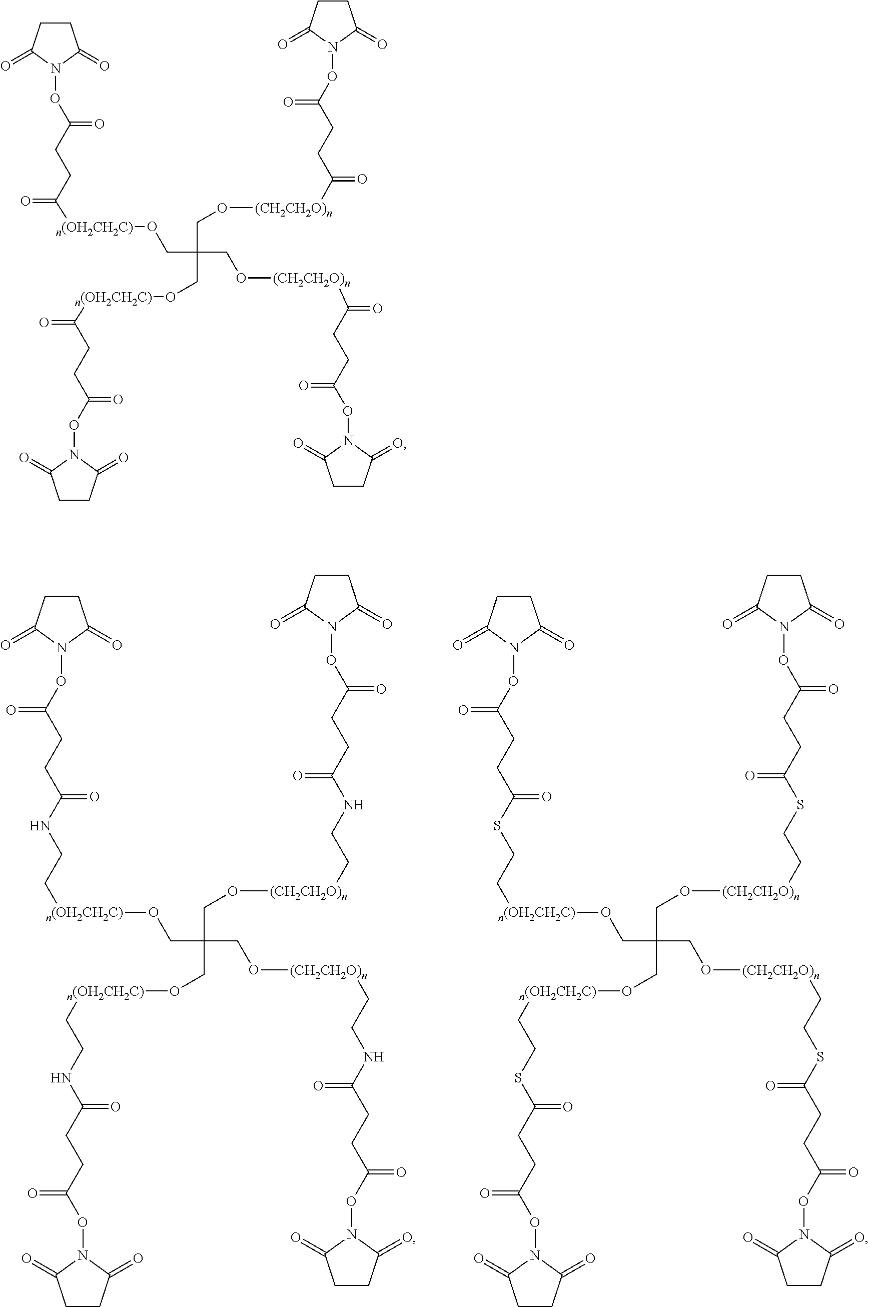

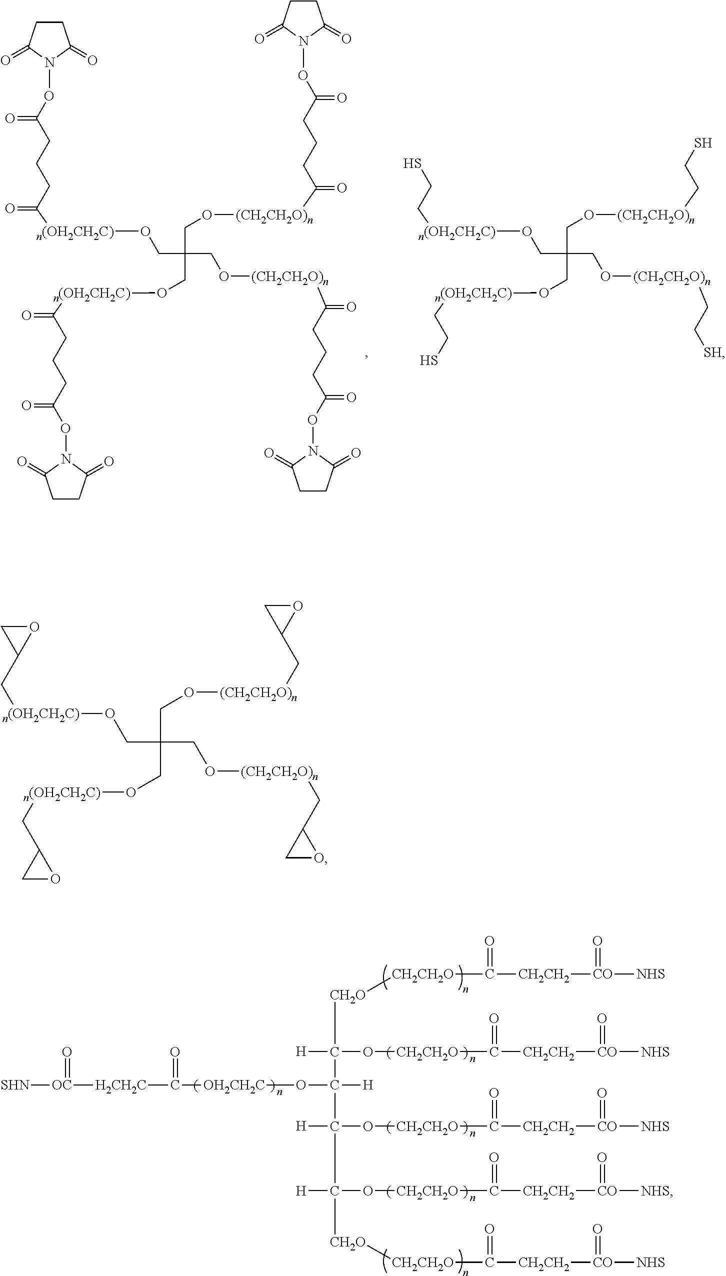

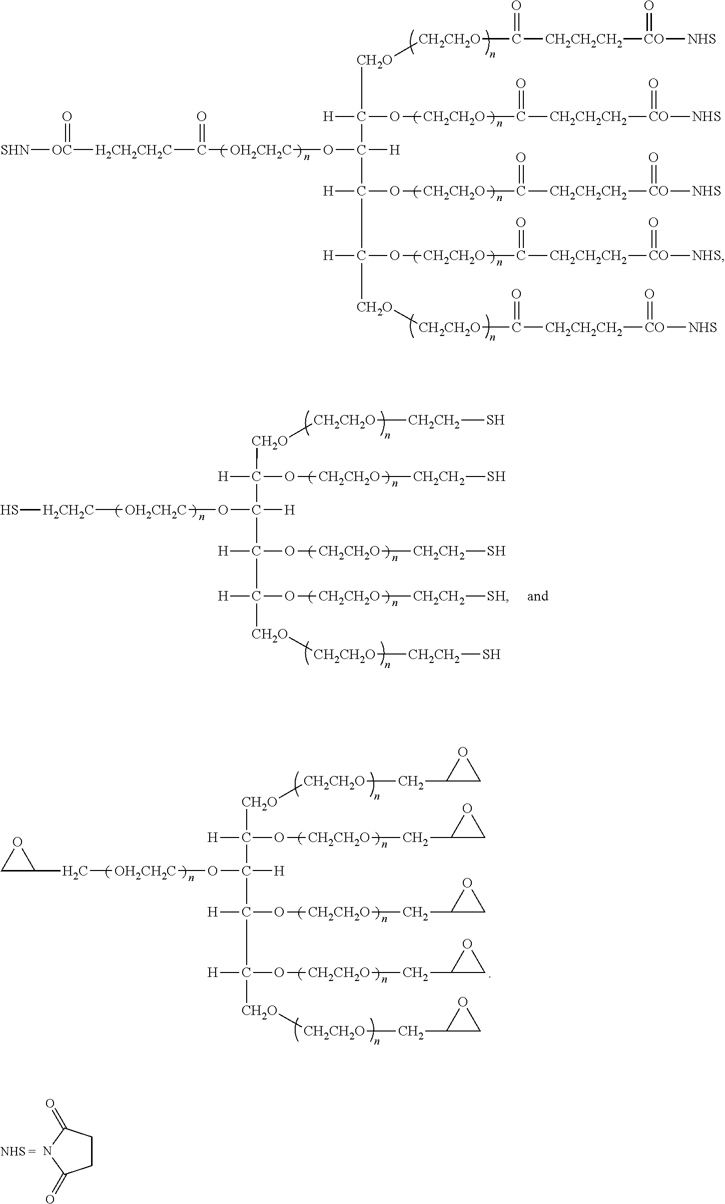

6. The method according to any one of claims 1 to 5, wherein the oral cavity composition is formulated in the form of granules or powder.
